(19) United States
(12) Patent Application Publication
(10) Pub. No.: US 2005/0267295 A1
(43) Pub. Date: Dec. 1, 2005

(54) Method for selective removal of a substance from samples containing compounds having nucleic acid structure

(76) Inventors: Makonnen Belew, Uppsala (SE); Jan Bergstrom, Balinge (SE); Rolf Berglund, Uppsala (SE); Lennart Soderberg, Uppsala (SE)

Correspondence Address: AMERSHAM BIOSCIENCES;PATENT DEPARTMENT, 800 CENTENNIAL AVENUE, PISCATAWAY, NJ, 08855, US

(21) Appl. No.: 11/076,477

(22) Filed: Mar. 9, 2005

Related U.S. Application Data
(63) Continuation-in-part of application No. 10/130,955, filed on Sep. 9, 2002.

(30) Foreign Application Priority Data
Nov. 25, 1999 (SE) 9904272-3

(51) Int. Cl. C07H 21/04

(52) U.S. Cl. 530/412; 536/25.4

(57) ABSTRACT

A method for purifying a desired substance by separating from each other a substance (I) from a substance (II), one of which is the desired substance, both of which have affinity for the same ligand structure, and wherein substance (I) is smaller than substance (II). The method comprising the steps of: (i) providing substances I and II in a liquid; (ii) contacting the liquid with an adsorbent which selectively adsorbs substance I; (iii) recovering the desired substance; The adsorbent has (a) an interior part which carries a ligand structure that is capable of binding to substances I and II, and is accessible to substance I, and (b) an outer surface layer that does not adsorb substance II, and is more easily penetrated by substance I than by substance II.

### CROSS-REFERENCE TO RELATED APPLICATIONS

This application is a continuation-in-part of U.S. patent application Ser. No. 10/130,955 filed Sep. 9, 2002, which is a filing under 35 U.S.C. § 371 and claims priority to international patent application number PCT/EP00/11677 filed Nov. 23, 2000 and published on May 31, 2001 as WO 01/37987 and also claims priority to patent application number 9904272-3 filed in Sweden on Nov. 25, 1999; the disclosures of which are incorporated herein by reference in their entireties.

### BACKGROUND OF INVENTION

The present invention concerns a method for purification of a desired substance comprising nucleic acid structure and comprises that a liquid sample containing a first substance (I) and a second substance (II) is contacted with a separation medium to which substance I has a stronger tendency to partition compared to substance II.

After the partitioning step, substance I is recovered from the adsorbent and/or substance II from the liquid, depending on which of them is to be purified. Finally, either or both of the substances may be further purified. For substances having nucleic acid structure two main principles have previously been used:
1. The separation medium has a firmly attached ligand structure to which substances I and II have different abilities to become bound to or desorbed from. Typically the ligand structure is an anion exchange group and the separation is based on ion exchange, e.g. ion exchange chromatography (IEC). Some basic publications are Cohn W. E. (in: Nucleic acids, Vol 1 pp 211-241 (1955), Chargaff & Davidsson (eds), Academic Press, New York); Hall et al (J. Mol. Biol. 6 (1963) 115-127); Bendich et al (J. Am. Chem. Soc. 77 (1955) 3671-3673); and Taussig et al (J. Chromatog. 24 (1957) 448-449).
2. The separation medium has a pore size permitting easier transport of substance I than of substance II within the pores. The separation is performed as a gel filtration (GF). Some basic articles are Hjerten (Biochim. Biophys. Acta 79 (1964) 393-); and Bengtsson et al (Biochim. Biophys. Acta 119 (1967) 399-); Öberg et al (Arch. Biochem. Biophys. 119 (1967) 504-509); and Loeb (Biochim. Biophys. Acta 157 (1968) 424-426).

Separation media which have an interior part and an outer surface layer with different separation functionalities (e.g. anion exchange groups and no anion exchanging groups, respectively) have been previously described and suggested for the separation of proteins, nucleic acids, carbohydrates, lipids etc. See WO 9839094 (Amersham Pharmacia Biotech AB) and WO 9839364 (Amersham Pharmacia Biotech AB). None of these publications discloses how to use separation media in which there are layers of different functionalities for purifying nucleic acids in order to overcome the disadvantages discussed below. Purification of nucleic acid vectors such as plasmids, virus and the like and specific problems associated therewith are not discussed.

Despite the innumerable reports published in this area during the past 30 years, it still remains a difficult task to separate negatively charged nucleic acids from each other and from other negatively charged components such as proteins.

This is partly due to the fact that the focus has changed from laboratory to large-scale processes. Thus it has become important to have processes that give high yields and high purity in a minimum of process steps in order to minimise production costs. Examples are various kinds of antisense drugs comprising synthetic oligonucleotides, recombinantly produced nucleic acids, such as nucleic acid vectors including viruses and plasmids, and recombinant proteins.

For compounds that comprise nucleic acid structure, individual process steps might increase the risk for conformational changes and irreversible denaturation/degradation, i.e. formation of contaminants, which are difficult to remove. This applies particularly to nucleic acids vectors, for instance plasmids. Covalently closed circular (CCC) plasmids (supercoiled), for instance, may easily be transformed to the open circular form, which shows a lowered efficacy for therapy.

Substances having nucleic acid structures bind strongly to anion exchangers and desorption often require conditions that can be harmful for the product, in particular nucleic acid vectors such as plasmids. It would be beneficial to use anion exchange material that combine strong binding, high capacity with mild conditions for desorption.

It is an objective is to provide methods for the purification of cellular components, which methods are improved with respect to (a) simplicity of operation, (b) increased purity and yield of a desired substance and (c) a reduction of the number of steps involved.

Another objective is to provide a method for separation of cellular components, such as proteins and/or peptides from cells and/or nucleic acids. A specific objective is to purify at least one protein from cells and/or cell debris of a cell culture or a cell lysate. A further objective is to provide such a method, which is useful at relatively high salt concentrations.

### SUMMARY OF THE INVENTION

We have now discovered that the separation methods for the separation of cellular components such as nucleic acids described in the introductory part can be improved if the adsorbent carries a shielding layer (lock, lid) which hinders passage of substance II into the interior part of the adsorbent matrix.

We have also discovered that there are advantages if the anion exchange ligand is selected to provide
(a) an enhanced binding via a mixed mode interaction, for instance involving hydrogen-bonding or other electron donor-acceptor interactions combined with a charge-charge interaction and/or
(b) milder desorption conditions by permitting decharging of the anion exchange ligands by a pH-switch (increase in pH) at moderate alkaline pH-values.

Especially in the purification of cell components such as proteins fron crude cell lysates, because of the cell properties, it is very advantageous to operate at slightly increased salt concentrations which ligands known as mixed mode or multimodal anion exchangers are capable of withstanding. For a reference to mixed mode anion exchangers, see e.g. U.S. Pat. No. 6,702,943 and WO 01/38228, which are hereby incorporated herein via reference.

### BRIEF DESCRIPTION OF THE FIGURES

FIG. 1A-C show the chromatograms resulting from Example 5, wherein the curve marked (XX) shows the absorbance at 600 nm and the curve marked (YY) shows the conductivity. 100% indicates the cell pulse injected to by-pass. FT indicates flow through (unbound cells) from the cell pulse injected to column. E indicates bound cells which can be eluted. 1A: A300 Base Matrix, starting buffer 20 mM Tris, pH 8.0. 1B: A300 ANX, starting buffer 20 mM Tris, 50 mM NaCl, pH 8.0. 1C: A300 ANX with dextran lid, starting buffer same as in B.

### DETAILED DESCRIPTION OF THE INVENTION

A first aspect of the invention is a method for purifying a substance from another cellular component, which has a different size but affinity for the same ligand structure as the substance to be purified. In other words the method means separation of two substances from each other (substance I and substance II) which differ in size but have affinity for a common ligand structure.

The method thus comprises the steps of:
(i) providing substances I and II in a liquid (sample);
(ii) contacting the liquid with an adsorbent which has a high selectivity for adsorbing substance I compared to substance II;
(iii) recovering the desired substance from the adsorbent as substance I and/or from the aqueous liquid as substance II;
   If necessary, either or both of the substances recovered in step (iii) is further purified.

The characteristic features of the inventive method are that the separation medium used has
(a) an interior part which
   carries a ligand structure which is capable of binding to both substances I and II, and
   is accessible to substance I, and
(b) an outer surface layer that does not substantially adsorb substance II, and is easier penetrated by substance I than by substance II.

This means that the outer surface layer is accessible to substances in the sample by convective mass transport, and that the interior part of the matrix is only accessible via diffusive mass transport. The outer surface layer may thus be considered as a border-layer limiting a convective environment from a diffusive environment.

The outer surface layer may be located to the outer surface of porous particles or to the surface of macropores within particles or within monoliths comprising both macropores and micropores. The pores, at least in the outer surface layer, have a molecular size cut-off value for influx of compounds corresponding to an apparent molecular size between the apparent molecular sizes (hydrodynamic radius) for substance I and substance II, respectively. This typically means that the pores in the outer surface layer are <1 μm. The interior part may have pores with molecular cut-off values that are the same as pores in the outer surface layer, or have pores that are larger or smaller than these pores. The interior part may also contain a combination of these pore sizes.

The expression “carries a ligand structure which is capable of binding to both substances I and II” means that each of the substances is capable of binding to the ligand structure if they have had access to it. It follows that the difference in selectivity between substance I and substance II for binding to the bead is primarily caused by the pore size of the outer surface layer and not by a difference in the affinity as such for the ligand structure.

The expression “is easily penetrated by substance I compared to substance II” means that substance I is transported substantially faster through the outer surface layer than substance II. This includes that substance II is completely excluded from the outer layer.

The expression “an outer surface layer that does not substantially adsorb substance II” means that at least the surface of the layer is essentially free from adsorptive ligand structures.

The outer surface layer may also contain repelling structures, e.g. structures of the same charge as substance I and II; hydrophobic structures in case substance II has a hydrophilic character that is incompatible with hydrophobic structures, etc. Repelling structures may improve the selectivity in transport through the outer surface layer. See WO 9839364 (Amersham Pharmacia Biotech AB).

The Sample.

The sample can be derived from different sources and prepared in various ways. It may be derived from a blood sample, tissue sample, cultured cells etc. It may be in the form of a crude cell extract or a cell lysate. It may also be a processed sample that has undergone centrifugation, filtration, ultrafiltration, dialysis, precipitation etc for removing particulate matters, proteins, certain fractions of nucleic acids, concentration, desalting etc. Thus, it is common practice to
(a) precipitate sample proteins before capturing and/or fractionating nucleic acids on an adsorbent,
(b) precipitate RNA, if a particular DNA fraction is to be isolated,
(c) reduce the ionic strength by desalting and/or diluting in case the sample is to be applied to an ion exchanger etc.

Other methodologies may also be applied in order to remove disturbing substances. In many cases the sample to be used in the instant invention is essentially free of particulate matters. For purification of plasmids the contents of contaminants in the sample typically are: protein ≦30 mg/ml, RNA ≦25 mg/ml. Endotoxin content may be >200 EU per ml, typically >40 000 EU per ml. Relative to total nucleic acid content, the plasmid may be present in quantities ≧3% (w/w). Depending on the objective for a particular purification process and the starting material, the levels may be significantly lower. Similar values apply in case the desired substance is some other kind of nucleic acid vector, for instance a virus.

The sample typically is aqueous.

Substances I and II and Apparent Molecular Size Cut-Off Value.

At least one of substances I and II has nucleic acid structure. The remaining substance may be some other compound as long as it comprises a structure that also is capable of binding to the ligand structure used. This means that the other substance may be a protein/polypeptide, an endotoxin, or a lipid, a detergent, a cell or a part thereof etc. Either or both of the substances may be a complex or conglomerate in which one or more components comprise nucleic acid structure while one or more other components comprise other structures. In important variants of the invention both substances I and II comprise nucleic acid structures (oligo- or polynucleotide structure). acid structure (oligo- or polynucleotide structure). Each of substance I or II may be mixtures of compounds.

Specific examples of substances, which have nucleic acid structure, are native and synthetic DNA and RNA including fragments and derivatives thereof having-two or more nucleotides linked in sequence. Linear and circular forms of nucleic acids, mRNA, tRNA, rRNA, genomic DNA etc are included. Still further examples are nucleic acid vectors such as viruses (including bacteriophages) and plasmids. Plasmids may be linear or circular. Circular forms include open circular (OC) forms and covalently closed circular (CCC) forms, i.e. supercoiled forms.

The apparent molecular size of a substance is determined by (a) its molecular weight, and (b) its shape under the conditions applied. The apparent size may thus change upon change of pH, ionic strength, type of salt and temperature. This is in particular true for biopolymers such as high molecular weight nucleic acids and proteins. Matching of pore sizes within the interior part and within the outer surface layer with apparent sizes of substances I and II is easily done by testing the molecular size exclusion behaviour of different interior parts and locks. It will also be possible to draw conclusions from the size exclusion behaviour of the substances concerned on various size exclusion separation media. Common knowledge from size exclusion chromatography applies.

By properly setting the molecular size cut-off value of the outer surface layer, step (ii) of the present invention will facilitate separations of substances in a sample into two fractions, which contain substances of apparent sizes above, respectively, below the molecular size cut-off value. One can thus envisage that the invention will render it possible to separate linear forms of DNA from circular forms of DNA, open circular forms from covalently closed circular forms, RNAs from plasmids, plasmids from genomic DNA, plasmids from plasmids, plasmids from endotoxins etc. Typically the most useful molecular size cut-off values for the purification of plasmids will be in the interval corresponding to the apparent molecular sizes for useful supercoiled plasmids, i.e. in the interval 1-10 kbp (kilo base pairs). This does not exclude that the cut-off value can be larger in case larger molecules are allowed to penetrate the interior part, for instance the interval may correspond to nucleic acid vectors containing from 1 to 40 kbp.

In the preferred mode of the instant invention, the molecular size cut-off value of the outer surface layer is set so that the desired substance is retained in the liquid (substance II), i.e. not transported to any significant extent into the interior part of the matrix. This principle has been found to be advantageous if the desired substance is substance II and is a nucleic acid vector, such as a virus or a plasmid. One of the main advantages is that the desired substance then does not need to go through an adsorption/desorption process that may reduce yield and cause denaturation/degradation of the substance.

One can envisage that it will be possible to set molecular size cut-off values that will make it possible to discriminate between high molecular weight genomic DNA and nucleic acid vectors. The smallest one (smallest apparent molecular size) will be bound to the interior part of the matrix while the largest one will be retained in the liquid.

Ligand Structures

The ligand structure (ligand) as such shall have affinity for both substances I and II. Since at least one of the substances comprises a nucleic acid structure, the most apparent ligand structures contain positively charged groups (anion exchanging groups). Anion exchanging groups in principle bind to any negatively charged species. Therefore, these kinds of ligand structures may be used in the instant invention for separating any negatively charged species from a substance comprising nucleic acid structure. The only demand is that the difference in apparent molecular size shall be sufficiently large.

One and the same matrix may contain two or more different ligands, for instance anion exchange ligands.

Illustrative examples of anion exchanging groups are primary, secondary, tertiary and quaternary ammonium groups that are linked via a spacer to a base matrix. Illustrative examples are —N⁺R₁R₂R₃ in which R₁₋₃ are hydrogen and/or hydrocarbon groups. The spacer is attached to the free valence of the —N⁺R₁R₂R₃ group. The carbon chains in R₁₋₃ may be interrupted at one or more location by an ether oxygen (—O—) or a thioether sulphur (—S—) or a secondary, tertiary or quaternary ammonium group (—N⁺R₄R₅—). The carbon chains may also be substituted by one or more —OR₆ or primary, secondary, tertiary or quaternary ammonium group (—N⁺R₇R₈R₉) in which R₄₋₉ are hydrogen or hydrocarbon groups. The groups R₁₋₉ may be identical or different. Hydrocarbon groups can be saturated, unsaturated or aromatic, and/or linear, branched or cyclic. R₁₋₉ is typically selected amongst hydrogen or C₁₋₁₀, preferably C₁₋₆, hydrocarbon groups that preferably are alkyl groups. R₁₋₉ may pair-wise, if appropriate, form five- or six-membered rings including the atom(s) to which the involved R groups are attached.

The preferred anion exchange ligands provide mixed mode interaction with the substance to be bound and/or allow for decharging by a pH-switch (increase in pH) at moderate alkaline pH-values. The ability of decharging means that the anion exchange ligands comprise primary, secondary and tertiary ammonium groups, with preference for those having pKa ≦10.5 or ≦10.0, i.e. typical primary or secondary ammonium groups. In the variants believed to be most preferred and as reduced to practice in the experimental part, essentially all anion exchange groups should comply with this criterion.

The term “the anion exchange ligand provides mixed mode interaction with the substance to be bound” refers to a ligand that is capable of providing at least two different, but co-operative, sites which interact with the substance to be bound. One of these sites gives an attractive type of charge-charge interaction between the ligand and the substance of interest. The second site typically gives electron donor-acceptor interaction including hydrogen-bonding.

Electron donor-acceptor interactions mean that an electronegative atom with a free pair of electrons acts as a donor and bind to an electron-deficient atom that acts as an acceptor for the electron pair of the donor. See Karger et al., An Introduction into Separation Science, John Wiley & Sons (1973) page 42. Illustrative examples of donor atoms/groups are:
(a) oxygen with a free pair of electrons, such as in hydroxy, ethers, carbonyls, and esters (—O— and —CO—O—) and amides,
(b) sulphur with a free electron pair, such as in thioethers (—S—),
(c) nitrogen with a free pair of electron, such as in amines, amides including sulphone amides, cyano,
(d) halo (fluorine, chlorine, bromine and iodine), and
(e) sp- and sp²-hybridised carbons.
   Typical acceptor atoms/groups are electron deficient atoms or groups, such as metal ions, cyano, nitrogen in nitro etc, and include a hydrogen bound to an electronegative atom such as HO— in hydroxy and carboxy, —NH— in amides and amines, HS— in thiol etc.

The distance between the donor or acceptor atom/group and the positively charged atom is typically 1-7 atoms, with preference for 2, 3, 4 and 5 atoms.

Examples of suitable anion exchange ligand structures may be found amongst those that contain primary, secondary and tertiary ammonium groups, typically containing no aromatic or unsaturated structures. Particularly preferred groups have one, two or more hydroxyl group or a primary, secondary or tertiary amino nitrogen on at least one carbon atom that is located at a distance of 2 or 3 atoms away from the amino nitrogen of the ammonium group. These 2 or 3 atoms are typically sp³-hybridised carbon atoms. One or more of these hydroxyl groups and amino nitrogens may or may not be present in the spacer. See also WO 9729825 (Amersham Pharmacia Biotech AB,=U.S. Pat. No. 6,090,288) which is hereby incorporated by reference. Such exemplary ligands structures, inclusive the ending of the spacer (bold) are:
—CHOHCH₂NH(CH₂)₃NH(CH₂)₃NH₂—CHOHCH₂NHCH₂CH₂CH₃
—CHOHCH₂NHCH₂CH₂NHCH₂CH₂NHCH₂CH₂NH₂—CHOHCH₂NH₂
—CHOHCH₂N [C(CH₂OH)₃](CH₂)₃NHC(CH₂OH)₃—CHOHCH₂NHCH(CH₂OH)₂
—CHOHCH₂NHCH₂ (CHOH)₄CH₂OH —CHOHCH₂NHC(CH₂OH)₃
—CHOHCH₂NHC(CH₃) (CH₂OH)₂ —CHOHCH₂N[(CH₂)₃NH₂]₂
—CHOHCH₂NHCH₂CHOHCH₂OH —CHOHCH₂NH [C(CH₃)₃]
—CHOHCH₂NHCH₂CH₂OH —CHOHCH₂N(CH₂CH₂OH)₂
   CHOHCHOH
—CHOHCH₂NH—CH CHCH₂OH
   O CHOH

The ligand structures of particular interest are those that, when bound to matrix, can adsorb substances at increased ionic strength compared to a conventional reference anion exchanger. In most cases this means that the preferred anion exchangers will exhibit an increased elution ionic strength compared to a conventional reference anion exchanger. This can be expressed in such a way that the maximum elution ionic strength in the pH range 2-14 for an anion exchanger (I) carrying an ammonium ligand structure as defined above should be higher than, in preferred cases ≧125%, in many cases ≧140%, such as ≧200% of the required elution ionic strength for a quaternary anion exchanger (II) with the ion exchanging group (CH₃)₃N⁺— (=Q-group; the same matrix, the same coupling group from the quaternary nitrogen and in towards the matrix, the same level of ligand concentration as for the anion exchanger (I) and measured at the same pH) for desorption of at least one of the proteins transferrin, ovalbumin 1, ovalbumin 2, β-lactoglobulin 1 and β-lactoglobulin 2. See WO 9729825 (Amersham Pharmacia Biotech AB).

According to another selection criterion suitable anion-exchangers may be found amongst those that have a maximal breakthrough capacity somewhere in the pH-interval 2-12 for at least one of the reference proteins: ovalbumin, conalbumin, bovine serum albumin, β-lactglobulin, α-lactalbumin, lyzozyme, IgG, soybean trypsin inhibitor (STI) which is ≧200%, such as ≧300% or ≧500% or ≧1000% of the corresponding breakthrough capacity obtained for a Q-exchanger (—CH₂CH(OH)CH₂N⁺(CH₃)₃. The support matrix, degree of substitution, counter-ion etc are essentially the same in the same sense as discussed above. The reference anion-exchanger is Q Sepharose Fast Flow (Amersham Pharmacia Biotech AB, Uppsdala, Sweden). This reference anion-exchanger is a strong anion-exchanger whose ligand and spacer arm structure are: —O—CH₂CHOHCH₂OCH₂CHOHCH₂N⁺(CH₃)₃.

Its chloride ion capacity is 0.18-0.25 mmol/ml gel. The base matrix is epichlorohydrin cross-linked agarose in beaded form. The beads have diameters in the interval 45-165 μm. The exclusion limit for globular proteins is 4×10⁶.

See further International Patent Applications (Amersham Pharmacia Biotech AB) based on SE application SE 9904197-2 with filing date Nov. 22, 1999. These International Patent Applications are hereby incorporated by reference.

Alternative ligand structures may be selected amongst nucleic acid structures complementary to at least part of the nucleic acid structure of substance I. The complementarity should be sufficient for permitting hybridisation between the ligand structure and substances I under the binding conditions applied. This kind of ligand structure requires that substance II also carries a nucleic acid structure that at least partially is essentially the same as in substance II. Poly-U and nucleic acid binding proteins are examples.

In case substances I and II also have other structures than nucleic acid structures and other negatively charged groups, ligand structures binding to these could also be used for capturing substance I selectively by the separation medium according to the instant invention.

The ligand structure is typically covalently linked to the matrix via a spacer as known in the field. The spacer may be an organic structure, which is hydrolytically stable under the pH conditions normally utilized for anion exchange adsorption, i.e. pH 2-14. The spacer typically lacks hydrolytically unstable structures, such as silane, carboxylic acid ester (—COO—) or carboxylic acid amide (—CONH—). The spacer is preferably a linear, branched or cyclic saturated or unsaturated hydrocarbon chain. The chain is optionally interrupted at one or more locations by an ether oxygen (—O—) or a thioether sulphur (—S—) and/or an amino nitrogen (—N⁺R₁₀R₁₁—) or substituted by one or more —N⁺R₁₂R₁₃R₁₄ groups or —OR₁₅ groups. R₁₀₋₁₅ are selected according to the same rules as for the other R groups discussed above. The ligand structure may also be bound non-covalently as long as the link is capable of withstanding the conditions used for adsorption/desorption.

As discussed in WO 9729825 (Amersham Pharmacia Biotech AB) insertion of primary, secondary or tertiary amine ligands is easily done by methods not giving rise to any significant amount of quaternary ammonium structures. This latter kind of structure is not dechargeable by a simple pH-shift.

The Interior Part of the Matrix

This part of the matrix is typically of the same type as commonly utilized within affinity adsorption such as chromatography. As discussed above the interior part may comprise both macropores and micropores.

The interior part is preferably hydrophilic and in the form of a polymer, which is insoluble and more or less swellable in water. Hydrophilic polymers typically carry polar groups such as hydroxy, amino, carboxy, ester, ether of lower alkyls (such as (—CH₂CH₂O—)ₙH, (—CH₂CH(CH₃)O—)ₙH, and groups that are copolymerisates of ethylene oxide and propylene oxide (e.g. Pluronics®) (n is an integer >0, for instance 1, 2, 3 up to 100). Hydrophobic polymers that have been derivatized to become hydrophilic are also included in this definition. Suitable polymers are polyhydroxy polymers, e.g. based on polysaccharides, such as agarose, dextran, cellulose, starch, pullulan, etc. and completely synthetic polymers, such as polyacrylic amide, polymethacrylic amide, poly(hydroxyalkyl vinyl ethers), poly(hydroxyalkylacrylates) and polymethacrylates (e.g. polyglycidylmethacrylate), polyvinylalcohols and polymers based on styrenes and divinylbenzenes, and copolymers in which two or more of the monomers corresponding to the above-mentioned polymers are included. Polymers, which are soluble in water, may be derivatized to become insoluble, e.g. by cross-linking and by coupling to an insoluble matrix via adsorption or covalent binding. Hydrophilic groups can be introduced on hydrophobic polymers (e.g. on copolymers of monovinyl and divinylbenzenes) by polymerization of monomers exhibiting groups which can be converted to OH, or by hydrophilization of the final polymer, e.g. by adsorption of suitable compounds, such as hydrophilic polymers.

The interior part can also be based on inorganic material, such as silica, zirconium oxide, graphite, tantalum oxide etc.

The interior part is preferably devoid of hydrolytically unstable groups, such as silan, ester, amide groups and groups present in silica as such.

In a particularly interesting embodiment of the present invention, the interior part is in the form of irregular or spherical beads with sizes in the range of 1-1000 μm, preferably 5-1000 μm.

The interior part may also be in the form of a porous monolith.

The ligand structures are introduced into the interior part by methods known in the field as suggested above under the heading “Ligand Structures”.

The required degree of substitution for ligand structures (density of ligand structures) will depend on ligand type, kind of matrix, compound to be removed etc. Usually it is selected in the interval of 0.001-4 mmol/ml matrix, such as 0.01-1 mmol. For agarose-based matrices the density is usually within the range of 0.1-0.3 mmol/ml matrix. For dextran based matrices the interval the interval may be extended upwards to 0.5-0.6 mmol/ml matrix.

The ranges given in the preceding paragraph refer to the capacity for the matrix in fully protonated form to bind chloride ions. “ml matrix” refers to the matrix saturated with water. The outer surface layer is included in the matrix in calculating these ranges.

The Outer Surface Layer

The outer surface layer must be penetrable by the liquid sample. For aqueous liquid this means that the outer surface layer should be built up of a hydrophilic polymer, of the same kind as discussed for the interior part.

There are different methodologies for creating the outer surface layer.
I. Coating the surface of a naked form of a porous particle or the surfaces of macropores of particles or of a monolith which have both macropores and micropores with a hydrophilic polymer. The apparent molecular size of the hydrophilic polymer should be selected such that it cannot significantly penetrate the pores that are aimed at being part of the interior. Preferably the hydrophilic polymer comprises hydrophilic groups as discussed above, e.g. is a polyhydroxy polymer such as polysaccharides in soluble forms (dextran, agarose, starch, cellulose etc).

The ligand structures may be introduced onto the interior part either before or after creation of the lock. The permeability for various substances of the outer surface layer produced in this way will be controlled by the concentration and size of the polymer in the solution used for coating. Subsequent to coating the outer surface layer may be stabilized by crosslinking within the layer as well as to the interior part. This methodology is described in detail in WO 9839094 (Amersham Pharmacia Biotech AB).
II. Starting from a naked hydrophilic base matrix of the type discussed under the heading Interior Part above and then specifically introducing the ligand structure into an interior part of the matrix leaving an outer surface layer devoid of ligand structure. It is preferred to select the porosity of the starting matrix such that substance I will have a facilitated transport compared to substance II within the matrix, i.e. the pore size of the interior part and the outer surface layer are essentially the same. This kind of methodology has been presented in Wo 9839364 (Amersham Pharmacia Biotech AB).

The lock medium used in the present invention may be in the form of particles/beads that have densities higher or lower than the liquid (for instance by introducing one or more density-controlling particles per matrix particle). This kind of matrix is especially applicable in large-scale operations for fluidised or expanded bed chromatography as well as different batch-wise chromatography techniques in non-packed columns, e.g. simple batch adsorption in stirred tanks. These kinds of techniques are described in WO 9218237 (Amersham Pharmacia Biotech AB) and WO 92/00799 (Kem-En-Tek/Upfront Chromatography) and can easily be adapted to the inventive concept by introducing a lock on the particles used.

Process Conditions

The conditions for running the inventive process are in principle the same as for conventional adsorption techniques, e.g. anion exchange chromatography.

For positively charged ligand structures this means that the matrix is first equilibrated to a suitable pH where the ligand structures are positively charged and an ionic strength that is well below the maximum ionic strength permitted for adsorption. This typically means that the ionic strength should be below the elution ionic strength for the particular combination of substance(s), anion exchanger and other conditions etc. The sample is then applied. After adsorption either or both of the liquid phase and the matrix are further processed with respect to substances I and II, respectively. Desorption of substance I from the matrix is accomplished by increasing the ionic strength of the liquid in contact with the matrix until substance I is eluted. In particular in case the ligand structure is the protonated form of a primary, secondary or tertiary amine group and/or substance I is a nucleic acid, desorption is preferably assisted by increasing the pH. An alternative method for desorption is to include a soluble ligand analogue in the liquid, i.e. a structure analogue that is able to compete with the ligand structure for binding to substance I. The presence of structure-breaking compounds in the liquid may also assist desorption. This in particular may apply in case the ligand structure contains one or more hydroxyl group or amino group at a carbon atom at 2 or 3 atoms distance from a charged primary, secondary or tertiary nitrogen of the ligand structure. Well-known structure breaking agents are guanidine and urea. See also WO 9729825 (Amersham Pharmacia Biotech AB).

The above-mentioned desorption principles may be combined as found approriate for a particular ligand structure and substance I

Changes in the composition of the liquid in contact with the matrix can be made in order to accomplish desorption of substance I either as a step-wise gradient or a continuous gradient with respect to pH and/or concentration of salt and/or other desorbing agents. If possible it is simplest to make the change in one step. Continuous gradients and stepwise gradients containing two or more steps have their primary use in case substance I has been bound to the matrix together with one or more additional substances. In these cases the desorption gradient may be used for desorbing the substances during different conditions thereby improving the purity of recovered substance I.

As indicated above either substance I or II may be further purified, for instance by so called polishing and or intermediate purification steps. After desorption, substance I may be further purified by additional capture steps either for capturing the desired substance or for capturing contaminants. If substance II is desired in purified form it may also be subjected to additional capturing step. The need for extra purification/polishing steps typically applies if the purity demand on the desired substance is high, such as for in vivo therapeutics. Such additional steps may involve adsorbtion/desorption of substance I or II to/from an anion exchanger, a cation exchanger, a reverse phase matrix, a HIC matrix (hydrophobic interaction chromatography matrix) etc. Size exclusion chromatography and adsorption/desorption on hydroxy apatite may also be used. There may also be one or more of the above-mentioned adsorption/desorption steps before the step, which utilizes a lock based affinity matrix.

For large-scale production of the desired substance as defined above it is of utmost importance to have selected matrix material, ligand and coupling chemistry that will permit desorption, regeneration and re-use of the adsorbent/separation medium. Re-use typically starts with regenerating and equilibrating the adsorbent after step (iii) whereafter the adsorbent is contacted as defined in step (ii) with a new batch of sample. The regeneration and equilibration is done as known in the field. In certain variants these two steps may coincide. This kind of cyclic use of the separation medium typically demands a cleaning step either before or after the regeneration step. A cleaning step may be present in each cycle, or every second, third, fourth, fifth etc cycle or whenever found appropriate.

A second separate aspect of the invention is the use of separation media, which carry the above-mentioned primary, secondary and tertiary ammonium groups and
(a) which are able to adsorb at an increased ionic strength as defined above and/or
(b) which have one, two or more hydroxyl groups and/or amino nitrogens at a distance of two or three sp³-hybridised carbon atoms from the ammonium groups,
   for the removal and/or purification of nucleic acid vectors.

In this aspect of the invention the ability to adsorb at an increased ionic strength, the kind of ligand and spacer, matrix features, such as porosity, matrix material, etc are as defined above. The matrix may be fully functionalized, or only functionalized in its interior as defined above. The amino nitrogens referred to are preferentially primary, secondary or tertiary amino nitrogens with sub-alternatives and preferences as discussed for the first aspect of the invention.

This separate aspect is based on our previous discovery that anion exchangers in which one, two or more hydroxyl groups and/or amino nitrogens are present at a distance of two or three sp³-hybridised carbons from a positively charged amine nitrogen enhances binding of substances to the anion exchangers. See WO 97298725 (Amersham Pharmacia Biotech AB). We have now recognized that this can give certain advantages when dealing with nucleic acid vectors. See above.

One variant of this aspect is the purification method defined in the first aspect.

In another variant the separation medium lacks the outer surface layer (including a lock). The base matrix carrying the ligand structure in this variant may be of the same construction as the interior part described above. This variant means that the separation medium is used in a conventional capture step, for instance as described in WO 9916869 (Amersham Pharmacia Biotech AB). The vector plus contaminating species such as RNAs are selectively adsorbed and desorbed. The full process may contain additional purification steps as defined above for the process utilizing a lock separation medium.

In both variants the sample which contains the nucleic acid vector may have been treated as known in the field in order to remove proteins and/or nucleic acids.

See for instance WO 9916869 (Amersham Pharmacia Biotech AB) and Ollivier and Stadler, Gene Therapy of Cancer (editors Walden et al), Plenum Press, New York (1998) 487-492 and GB patent application 9927904.4 and corresponding International patent Application.

The invention is further defined in the appended claims. The invention will now be verified and illustrated with a number of patent examples.

### EXAMPLES

Below, the present invention will be illustrated by way of examples. However, the present examples are provided for illustrative purposes only and should not be construed as limiting the present invention as defined by the appended claims. All references given below and elsewhere in the present specification are hereby included herein by reference.

Synthesis

### Example 1

### Anion Exchanger in Particle Form with a Lock on the Particles

A. Allylated crosslinked agarose particles (allylated base matrix). Cross-linked agarose (90 μm particles) prepared by reaction between epichlorohydrin and agarose in the presence of NaOH according to Porath et al (J. Chromatog. 60 (1971) 167-77 and U.S. Pat. No. 3,959,251) was reacted with allylglycidyl ether with NaOH as a base to a allyl level (CH₂═CHCH₂OCH₂CHOHCH₂—) of 0.18-0.30 mmole/ml). This base matrix has a porosity which is similar to Sepharose 4B FF (Amersham Pharmacia AB, Uppsala, Sweden).

B. Introduction of a lock on allylated crosslinked agarose particles. 25 g vacuum drained allylated particles from A with an allylic content of 0.29 mmol/ml gel was charged together with 0.6 g anhydrous sodium acetate and 50 ml de-ionized water in 100 ml beaker fitted with a propeller stirrer. 0.18 ml bromine was added drop-wise under rapid stirring.

The brominated gel was then washed with plenty of de-ionized water and vacuum drained on a glass filter funnel. Gel and water were charged in a three-necked round flask. The water was added to a total weight of 50 g water and gel.

2 g Sodium hydroxide and 0.03 g sodium borohydride were then added and the temperature was raised to 60° C. After 21 h, 6 g thioglycerol was added in order to neutralise possible unhydrolysed epoxides. The reaction mixture was stirred for another 4 h at 60° C. The reaction was stopped by washing the gel on a glass filter funnel with water. A small amount acetic acid was added directly in the glass filter funnel and the slurry was made slightly acidic. A last wash with plenty of de-ionized water was carried out.

The remaining allylic content was determined to 0.21 mmol/ml.

C. Introduction of anion exchange ligand on lock beads prepared from allylated crosslinked agarose particles. 15 ml vacuum drained particle from B above, 0.63 g anhydrous sodium acetate and 100 ml de-ionized water was charged in 250 ml beaker fitted with a propeller stirrer.

Bromine (0.20 ml) was added drop-wise under rapid stirring.

The gel was washed with plenty of water. After vacuum draining on a glass filter funnel the gel was charged in a three necked 100 ml round flask already containing 22.5 g TRIS (NH₂C[CH₂OH]₃) and 22.5 g water.

The reaction was carried out at 60° C. over night 22.5 h.

The gel was then washed with a few bed volumes of water before pH was adjusted to about 7. Another washing step using plenty of water was carried out.

The material was sieved on a 45 μm sieve in order to get rid of small and crushed beads. The material left on the sieve was used as column packing in the chromatography experiments.

The total chloride ion capacity was determined to 0.08 (0.076) mmol/ml gel.

### Example 2

### Reference Matrix Without Lock (Naked Matrix) Functionalized with Tris Ligand

A. Allylated crosslinked agarose particles (allylated base matrix). This base matrix was prepared in the same way as in Example 1A. The allyl-ligand density was determined to 0.26 mmol/ml matrix.

B. Coupling of Tris (tris(hydroxymethyl) amine). 10 ml vacuum drained allylated gel from example 2A, 1.2 g sodium sulfate and 50 ml distilled water was mixed in 100 ml beaker fitted with a propeller stirrer. Bromine was added dropvise under rapid stirring until the slurry turned permanently yellow.

The gel was washed with plenty of water. After vacuum draining on a glass filter funnel the gel was transferred to a three necked 25 ml Bellco flask with a hanging magnetic stirrer which already contained 15 g Tris and 15 g distilled water. The reaction was carried out at 60° C. over night.

The pH of the reaction mixture was adjusted to 7 with dilute hydrochloric acid. A washing step using plenty of water (more than 100 ml) was carried out.

The final product had a ligand density (Ion Exchange Capacity) was 0.17 mmol/ml

Chromatography

### Example 3

### Chromatographic Experiments with Purified Plasmids

I. Materials

Separation media: Lock particles according to example 1 (separation medium A) and particles without lock according to example 2 (separation medium B).

Plasmid preparation: E. coli cells harbouring plasmid PXL 2784 (size=6.3 kbp) were lysed according to the standard alkaline lysis method of Birnboim (Birnboim et al., Nucleic Acids Res. 7 (1979) 1513-1523; and Birnboim, Meth. Enzymol. 100 (1983) 243-255). The sample was not treated with RNAse.

The purified plasmid PXL 3096 (2.5 kbp) was purified by using essentially hydroxy apatite chromatography while PXL 2784 (6.3 kbp) was prepared here in Uppsala using the Qiagen Kit (Qiagen) which meant RNAse treatment.

Equilibration buffer (A): 10 mM Tris-HCl, 1 mM EDTA, pH 8.0

Elution buffer (B): 1 M NaCl in Buffer A, pH 8.0

II. Chromatography

A column (HR 10/3 (Amersham Pharmacia Biotech AB, Uppsala, Sweden) containing separation medium A or B (bed volume 2.4 ml) was equilibrated at a flow rate of 30 cm/h. Then freshly prepared, and clarified, alkaline lysate (2 ml, containing about 50-80 μg of plasmid DNA) was applied. The column was then washed with: (i) 3 CV of the equilibration buffer to elute unbound material and, (ii) 3 CV of Buffer B to elute bound material. Fractions were pooled directly as they emerged from the column. When deemed necessary, the column was washed with 2 CV of 1 M NaOH followed with 3 CV of water.

III. Electrophoretic Analysis

This was performed on a 1% agarose gel (for nucleic acids) using a “sub-marine” electrophoresis assembly (GNA-100, 8 wells, 5×1 mm) and EPS 500/400 power supply (Amersham Pharmacia Biotech AB). A standard TBE buffer was used for electrophresis. The gels were stained with ethidium bromide and visualized by a UV lamp.

Results:

Separation medium A. Chromatography of purified plasmids on anion exchange particles with a lock. About 70 μg each of the purified plasmids PXL 3096 (2.5 kbp) and PXL 2784 (6.3 kbp) were chromatographed on the lock particles according to the procedure outlined above. The results showed that none of these 2 plasmids was bound to the column indicating that the porosity of the lock or polymer shield is such that diffusion of these macromolecules into the charged outer or inner surfaces of the anion-exchange particles is blocked. In effect, the plasmids are eluted in the void volume of the column and the lock medium acts as a passive molecular sieve.

Separation medium B. Chromatography of purified plasmids on anion exchange particles without a lock. The above experiment was repeated using particles without a lock according to example 2 under identical experimental conditions. The results showed that both plasmids are bound to this separation medium. This is because the plasmids have access to at least the charged outer surfaces of the anion-exchanger. The results also showed that the step-wise elution of the bound plasmids leads to their separation into at least 2 sub-fractions. The nature of these sub-fractions is not yet established and will be a topic for future investigations.

These results thus provide strong proof that the lock concept in media construction works in “real life situations” to solve one of the most difficult separation problems in biochemistry.

### Example 4

### Chromatography of Clarifed Alkaline Lysate (CAL) on a Lock Medium

This has been performed under varying experimental conditions.

For purposes of clarity, the results obtained will be presented in 3 separate sections.

I. Effect of De-Salting

2 ml of the clarified alkaline lysate (CAL) (sample A) containing the 6.3 kb plasmid was applied to the column without any further treatment and eluted according to the procedure outlined under “Experimental” in example 3. The experiment was repeated using 10 ml of de-salted CAL (which is equivalent to ca. 5 ml of the crude plasmid extract due to dilution during de-salting) (sample B). Desalting was performed on a PMl0 membrane (Amicon, U.S.A.) in a stirred cell using nitrogen gas to generate constant pressure. The material used was the same as in example 3 except for the equilibration and elution buffers.

Equilibration buffer (A): 10 mM Tris-HCl, 1 mM EDTA, pH 8.0

Elution buffer (B): 1 M NaCl in buffer (A), pH 8.0

Results:

The plasmid was in both cases eluted in the unbound fraction.

The bound fraction eluted as a broad peak (5-10 column volumes (CV)). The recovery in A260 was about 80%. The gel electrophoretic pattern obtained as described in example 3.III showed in both cases that the unbound fractions contained exclusively the plasmid while the bound fraction contained the RNA impurities. The unbound fraction for sample B showed a streaking band apparently because the plasmid might have been damaged when it was desalted. The unbound fraction for sample A seemed to contain a small amount of RNA possibly due to the high salt concentration in the sample resulting in a decreased adsorption capacity for the RNA. The following conclusions are consistent with the results obtained above:
1. The unbound fraction contains the plasmid DNA while the bound fraction contains RNA.
2. The bound fraction is eluted in a broad peak. The broadness of the peak may be due to a diffusion barrier created by the lock.
3. De-salting of the sample might be necessary to increase the adsorption capacity of the medium for RNA and other impurities.
4. The recovery in A₂₆₀ is ca. 80% indicating that some of the impurities are strongly bound and may require a cleaning step, e.g. washing with 1 M NaOH, to be completely eluted.
   II. Effect of pH

Anion exchange media containing the ligand used in Example 1 has its “optimum binding strength” for most anionic proteins at ca. pH 5.5-7.0. It is also virtually uncharged at around pH 9.0. See WO 9729825. One would therefore expect an efficient de-sorption of bound molecules from this adsorbent at approx. pH 9.0 or higher pH. The following experiment was therefore performed to establish/reject the validity of the above findings to plasmid DNAs. Except for the sample and the buffers used the chromatographic experiment was the same as in example 3.III.

Sample: Clarified crude alkaline lysate (pH=5.3). Not de-salted.

Equilibration buffer (A): 50 mM sodium acetate, 1 mM EDTA, pH 5.5

Elution buffer (B): 10 mM Tris-HCl, 1 mM EDTA, pH 9.1

Results:

Based on the chromatogram and the gel electrophoretic pattern it was that the plasmid eluted similarly to previous experiments in the unbound fraction and RNA in the bound fraction (in about 10 CV). The recovery in A₂₆₀ is about 80%.

Conclusions

1. As expected, the impurities were bound more strongly at pH 5.5 than at pH 8.0 just as in the case with anionic proteins. This might indicate that the separation medium used has a higher adsorption capacity for RNA (and possibly other impurities) at pH 5.5 and possibly even at pH 6.5.
2. The slower de-sorption kinetics might reflect a slow titration of the ion-exchanger from pH 5.5 to pH 9. If this is the case, and since the bound fraction comprises unwanted impurities, one can speed up the process by washing the column with 0.5 M NaOH. This has the added advantage that even strongly bound solutes might be eluted.
   III. Effect of the Unfunctionalised Outer Surface Layer (Lock).

The material and the procedure in this experiment were similar to example 3.

Separation medium: According to example 2, i.e. medium without lock.

Sample: 5 ml of the clarified alkaline lysate (pH adjusted to 6.4). Not de-salted.

Equilibration buffer (A): 20 mM sodium phosphate, 1 mM EDTA, pH 6.4

Elution buffer (B): 20 mM Tris-HCl, 1 mM EDTA, 0.5 M NaCl, pH 9.2

Procedure: See example 3.

The bound and unbound fractions were analysed by gel electrophoresis as described in example 3.

Results:

The unbound fraction eluted as a broad peak in about 15 CV. The unbound fraction contained exclusively plasmid DNA while the bound fraction contained both plasmid DNA and RNA.

### Example 5

### Protein Purification Without Cell Adhesion to Lock (Lid) Medium

In this example, the protein binding capacity of the medium was first tested in packed beds. To verify that lock media can be used according to the invention to avoid cell adhesion to the adsorbent, a subsequent test was run in expanded bed adsorption (EBA) mode, which is a commonly used chromtography mode for unclarified cell extracts.

I. Capacity

The dynamic breakthrough capacity (10% breakthrough capacity, QB10%) was determined for a comparative non-lid media (Streamline A300: agarose base matrix with steel fillers) and for the new EBA media prototypes (agarose base matrix with steel fillers and lids, prepared as previously described) at different linear flow rates (300 and 600 cm/h) in packed bed (2 ml in HR5/10 columns) using an AKTAexplorer 10 chromatography system and the UNICORN software (both Amersham Biosciences, Uppsala, Sweden). The reference media and the lid media are both provided with anion exchange groups ANX (Diethyl aminopropyl), in the lid media case said ligands are present in the interior part of the particles. A model protein solution (2.5 mg human IgG/ml in buffer A, 50 mM glycin, pH 9.0) was pumped through the UV-monitor (bypass mode) in the system until the UV-absorbance was stable, and this absorbance was equal to 100% breakthrough. The column was equilibrated with 5 column volumes (CV) of buffer A. Sample was applied until the UV-signal reached 15% (of the 100% level previously determined). The unbound sample was washed out with 3 CV buffer A, and the adsorbed protein was eluted with buffer B (50 mM glycin, 1 M NaCl, pH 9.0). Thereafter the column was cleaned with 1 CV of 1 M NaOH, and re-equilibrated with 8 CV buffer A. Even though the protein binding capacity decreased some cases, this test still shows that they are capable of protein binding to commonly satisfactorily levels.

TABLE 1

| | QB10%, 300 cm/h | QB10%, 600 cm/h |
|---|---|---|
| Prototype | (mg/ml) | (mg/ml) |
| ANX ref. (no lid) | 29.4 | 23.5 |
| ANX w. lid | 18.1 | 10.4 |
| ANX w. lid(PEG 2000) | 28.8 | 16 |
| ANX w. lid (OH-type) | 37.7 | 24.7 |

II. Cell Adsorption Measurement

Cell adsorption measurements were performed in expanded bed mode (EBA) using an AKTAexplorer chromatography system and the UNICORN software, as described above. 9 g of the EBA media prototypes (as previously above) were transferred to C 10/40 columns, equipped with two AC 10 adaptors with 80 μm net rings. This resulted in a sedimented bed height of approximately 10.5 cm. The columns were run in expanded bed mode, i.e. as a fluidised bed with upwards flow, at a flow rate of 4.3 ml/min (320 cm/h) resulting in an expanded bed height of 24-30 cm, and a space of approximately 3 cm between the upper adaptor and the expanded bed. The starting buffer consisted of 20 mM Tris-HCl, pH 8.0, containing 0-250 mM NaCl. The sample consisted of baker's yeast or E. coli cells resuspended in the starting buffer to an OD_{600 nm}=0.5 (approximately).

The volumes below are approximate, depending on when the absorbance has returned to baseline level. The column was always equilibrated with starting buffer before starting the method.

Starting conditions: Absorbance at 600, 700, and 280 nm recorded; Flow rate 4.3 ml/min.
0-5 ml Starting buffer, 20 mM Tris-HCl, pH 8.0, with 0.250 mM NaCl, through by-pass, for stable zero baseline. Fill Superloop with 8 ml of cell suspension (no bubbles) immediately prior to . . .
5-20 ml . . . switching the injection valve from Load to Inject.

Cell suspension injected through by-pass. Wait until the A₆₀₀ has returned to baseline before switching the valve back to Load.
20-25 ml Wash with starting buffer until A₆₀₀ returns to baseline.
25-70 ml Switch from by-pass to the desired column position. Allow bed to expand to stable bed height. Once the bed height is stable, fill SuperLoop with 8 ml of cell suspension (no bubbles), immediately prior to . . .
70-78 ml . . . switching the injection valve from Load to Inject. Cell suspension injected onto the column. Switch the injection valve back to Load immediately once the SuperLoop is empty.
78-115 ml Wash column with starting buffer until A₆₀₀ returns to baseline.
115-160 ml Elute the cells from the column with 20 mM Tris-HCl, 1 M NaCl, pH 8.0, until A600 returns to baseline.
160 ml End method.

The 160 ml-method described above takes 37 minutes for A300 media (flow rate 4.3 ml/min). The equilibration of columns to a different NaCl concentration (different % B), and Cleaning-In-Place (CIP), takes much more time, and is not included here.

The A₆₀₀ area of the chromatogram is evaluated using the UNICORN software. The A₆₀₀ area of the first 8 ml cell suspension pulse (by-pass) is set to 100%. Cells from the second, identical cell pulse (column) can pass through the column (flow through), or bind. Bound cells are eluted with 20 mM Tris-HCl, 1 M NaCl, pH 8.0. The A₆₀₀ areas of the flow through and eluted cells are evaluated and compared to the area of the first by-pass cell pulse (100% area). Ideally, in case of no cell-ligand interactions, one should expect approximately 100% of unbound cells, and 0% eluted cells. And in case of strong cell-ligand interactions, one should expect 0% unbound cells, and 100% bound cells. It is important that the same cell suspension is used for both 8 ml injections (one in by-pass, one on column) for correct comparison of the areas.

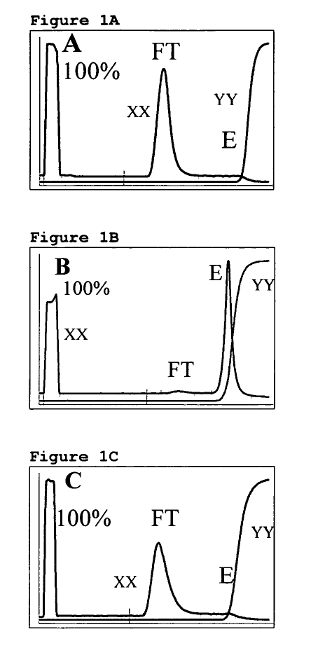

The results are presented in the chromatograms shown in FIG. 1, from which it appears that no cell adsorption was detected to the base matrix (Streamline A300), see FIG. 1A. All the cells bound to media with ANX ligand and no lid (FIG. 1B) using 20 mM Tris, 50 mM NaCl, pH 8.0 as starting buffer. This cell adsorption could be reduced by the lid-type surface modifications (FIG. 1C, compare to 1B). The protective ability of the different lid prototypes varied, depending on type of modification (e.g. dextran, PEG, OH), synthesis method used, and thickness of the lid. It was usually advantageous to include some NaCl (25 or 50 mM) in the starting buffer to completely eliminate cell adsorption (FIG. 1C).

Evaluation table: The area of the by-pass cell pulse is by default set to 100% (peak area in mAUxml/100% in table).

The areas of the FT and E peaks are evaluated, and their relative amounts calculated by dividing the FT and E peak areas with the 100% peak area. The yield is the % of recovered cells (% FT+% E).

TABLE 2

| Chrom. medium | 100% | FT | E | Yield |
|---|---|---|---|---|
| A: A300 Base | 675/100% | 667/99% | 0/0% | 99% |
| Matrix | | | | |
| B: A300 ANX | 673/100% | 24/3% | 631/94% | 97% |
| C: A300 ANX lid | 674/100% | 641/95% | 7/1% | 96% |

It is apparent that many modifications and variations of the invention as hereinabove set forth may be made without departing from the spirit and scope thereof. The specific embodiments described are given by way of example only, and the invention is limited only by the terms of the appended claims.

1. In a method for purifying a desired substance by separating from each other a substance I from a substance II, one of which is the desired substance, both of which have affinity for the same ligand structure, and wherein substance I has a smaller size than substance II, said method comprising the steps of:
(i) providing substances I and II in a liquid (sample);
(ii) contacting the liquid with an adsorbent which has a high selectivity for adsorbing substance I compared to substance II;
(iii) recovering the desired substance from the adsorbent as substance I or from the aqueous liquid as substance II;
(iv) further purifying, if necessary, the substance recovered in step (iii);
the improvement comprising suing as the adsorbent, a material having
(a) an interior part which
carries a ligand structure that is capable of binding to substances I and II, and
is accessible to substance I, and
(b) an outer surface layer that does not substantially adsorb substance II, and is more easily penetrated by substance I than by substance II.

2. The method of claim 1, wherein the outer surface layer is penetrable by substance I but not by substance II.

3. The method of claim 1, wherein the ligand structure includes a positively charged group.

4. The method of claim 3, wherein the positively charged group is selected from the group consisting of primary, secondary and tertiary ammonium groups.

5. The method of claim 3, wherein the positively charged group is a mixed mode anion exchanger.

6. The method of claim 1, wherein the outer surface layer is essentially free of ligand structures.

7. The method of claim 1, wherein substance I is the desired substance.

8. The method of claim 1, wherein substance I is a protein.

9. The method of claim 1, wherein substance II includes a cell and/or cell debris.

10. The method of claim 1, wherein both substances I and II comprise nucleic acid structures.

11. The method of claim 1, wherein the adsorbent is in an expanded bed.
